SEQUENCE LISTING (2 sequences)

SEQ ID NO: 1 - protein, 28 aa, Homo sapiens
  MISC_FEATURE (1)..(1): Terminal Amine
GHRPLDKKREEAPSLRPAPPPISGGGYR

SEQ ID NO: 2 - protein, 28 aa, Homo sapiens
  MISC_FEATURE (1)..(1): Terminal Amine
GHRPLDKKREEAPSLRPAPPPISGGGYR

(19) United States
(12) Patent Application Publication
(10) Pub. No.: US 2009/0081192 A1
(43) Pub. Date: Mar. 26, 2009

(54) METHODS OF SCREENING FOR COMPOUNDS HAVING ANTI-INFLAMMATORY ACTIVITY

(75) Inventors: Peter PETZELBAUER, Wien (AT); Rainer HENNING, Uetliburg (CH); Sonja REINGRUBER, Wien (AT); Waltraud PASTEINER, Pyhra (AT); Marion GROGER, Wien (AT)

Correspondence Address: Workman Nydegger;1000 Eagle Gate Tower, 60 East South Temple, Salt Lake City, UT, 84111, US

(73) Assignee: FIBREX MEDICAL RESEARCH & DEVELOPMENT GMBH, Wien (AT)

(21) Appl. No.: 11/860,488

(22) Filed: Sep. 24, 2007

(51) Int. Cl. C12Q 1/34; A61K 39/395; A61K 38/02; C12Q 1/48; C12Q 1/00; A61P 9/00

(52) U.S. Cl. 424/130.1; 435/18; 435/15; 435/4; 514/2

(57) ABSTRACT

A method screens compounds out of the group consisting of proteins, peptides, peptidomimetics, antibodies and small organic molecules that increase the activity of the protein Rac1 by virtue of their binding to the extracellular portion of this protein. The method includes the steps of: contacting a confluent layer of cultured endothelial cells with at least one of test compounds, lysing the endothelial cells with a lysation buffer, and measuring the amount of Rac1 activity.

### FIELD OF THE INVENTION

The present invention relates to the field of drug screening. More specifically, the present invention relates to methods for screening, identification and characterization of compounds, i.e. proteins, peptides, peptidomimetics, antibodies and small molecules, which bind to vascular endothelial (VE)-cadherin and influence certain signalling processes that are mediated by VE-cadherin. These compounds can be used to prevent the opening of endothelial adherens junctions between endothelial cells and certain morphological changes of endothelial cells as a consequence of these events. Compounds with these characteristics are useful for the treatment of all diseases, where inflammatory responses, vascular leak and endothelial dysfunction play a role. They can also prevent formation of new blood capillaries and are therefore useful for the treatment of cancer.

### BACKGROUND OF THE INVENTION

The endothelial layer which seamlessly covers the inside of all blood vessels, has a very important barrier function, preventing blood constituents such as blood borne substances, cells and serum from entering the underlying tissue. The barrier function is tightly regulated through a number of homo- and heterotopic interactions between molecules on neighbouring endothelial cells as well as similar interaction with molecules on circulating blood cells. The breakdown of this barrier function leads to severe physiological consequences and injury to the underlying tissue. It is involved in the pathogenesis of inflammatory diseases, edema formation as well as angiogenesis, for instance, but not limited to ischemia reperfusion injury caused by for instance myocardial infarction or organ transplantation, systemic inflammatory response syndrome (SIRS) as a sequel of trauma/resuscitation and septicemia, macula degeneration in the eye and in cancer progression. It is therefore desirable to identify compounds which are able to maintain the integrity of the endothelial adherens junction. These compounds can be used to treat or prevent these disease processes.

### Endothelial Barrier Dysfunction

Endothelial dysfunction can be manifested in a number of ways, for example as an imbalance between the release of relaxant and contractile factors, the release of anti- and pro-coagulant mediators, or as a loss in barrier function (Rubanyi, Journal of Cardiovascular Pharmacology 22, S1-14.1993; McQuaid & Keenan Experimental Physiology 82, 369-376 (1997)). Such dysfunction has been associated with numerous pathological conditions, including hypercholesterolemia, hypertension, vascular disease associated with diabetes mellitus, atherosclerosis, septic shock and the adult respiratory distress syndrome (Sinclair, Braude, Haslam & Evans, Chest. 106:535-539 (1994); Davies, Fulton & Hagen, Br J. Surg. 82:1598-610 (1995).

One of the principal abnormalities associated with acute inflammatory disease is the loss of endothelial barrier function. Structural and functional integrity of the endothelium is required for maintenance of barrier function and if either of these is compromised, solutes and excess plasma fluid leak through the monolayer, resulting in tissue oedema and migration of inflammatory cells. Many agents increase monolayer permeability by triggering endothelial cell shape changes such as contraction or retraction, leading to the formation of intercellular gaps (Lum & Malik, Am. J. Physiol. 267: L223-L241 (1994). These agents include e.g thrombin, bradykinin and vascular endothelial growth factor (VEGF). Endothelial cell contraction resembles the regulation of actin-myosin interaction in smooth muscle cells, but occurs over a longer time scale and is more properly described as a contracture. The mechanism of this contraction is thought to involve increases in intracellular Ca2+ concentrations, activation of myosin light chain kinase, phosphorylation of myosin light chain and reorganization of F-actin microfilaments. Retraction is a more passive process, is independent of myosin light chain kinase and involves protein kinase C (PKC)-stimulated phosphorylation of actin-linking proteins critical for maintaining cell-cell and cell-matrix interactions (Lum & Malik, Am. J. Physiol. 267: L223-L241 (1994).

Hyperpermeability of the blood vessel wall permits leakage of excess fluids and protein into the interstitial space. This acute inflammatory event is frequently allied with tissue ischemia and acute organ dysfunction. Thrombin formed at sites of activated endothelial cells (EC) initiates this microvessel barrier dysfunction due to the formation of large paracellular holes between adjacent EC (Carbajal et al, Am J Physiol Cell Physiol 279: C195-C204, 2000). This process features changes in EC shape due to myosin light chain phosphorylation (MLCP) that initiates the development of F-actin-dependent cytoskeletal contractile tension (Garcia et al, J Cell Physiol. 1995; 163:510-522 Lum & Malik, Am J Physiol Heart Circ Physiol. 273(5): H2442-H2451. (1997).

The signalling mechanism of this contractile process involves the proteolytic cleavage and activation of the thrombin receptor. This receptor is coupled to heterotrimeric G proteins of the Gq family that stimulate phospholipase Cβ, release D-myo-inositol 1,4,5-trisphosphate, mobilizing Ca ions from intracellular stores. The subsequent rise in intracellular Ca ion concentration activates Ca ion-calmodulin-dependent MLC kinase, which phosphorylates serine-19 and threonine-18 of MLC (Goeckeler & Wysolmerski, J. Cell Biol. 1995; 130:613-627). MLCP initiates myosin Mg ion-ATPase activity, causing the binding of myosin to F-actin and subsequent actomyosin stress fiber formation (Ridley & Hall, Cell, 70, 389-399 (1992). The phosphorylation of MLC converts the soluble folded 10S form of non-muscle myosin II to the insoluble unfolded 6S form. This process is characterized by reorganization of myosin from a diffuse intracellular cloud to punctuate spots and ribbons associated with large bundles of F-actin (Verkhovsky et al, The Journal of Cell Biology, 131, 989-1002 (1995). The final consequence is a persistent shape change of endothelial cells and a disruption of the barrier function.

### Vascular Endothelial (VE)-Cadherin

Thrombin-induced endothelial hyperpermeability may also be mediated by changes in cell-cell adhesion (Dejana J. Clin. Invest. 98: 1949-1953 (1996). Endothelial cell-cell adhesion is determined primarily by the function of vascular endothelial (VE) cadherin (cadherin 5), a Ca-dependent cell-cell adhesion molecule that forms adherens junctions. Cadherin 5 function is regulated from the cytoplasmic side through association with the accessory proteins beta-catenin, plakoglobin (g-catenin), and p120 that are linked, in turn, to alpha-catenin (homologous to vinculin) and the F-actin cytoskeleton.

VE-cadherin has emerged as an adhesion molecule that plays fundamental roles in microvascular permeability and in the morphogenic and proliferative events associated with angiogenesis (Vincent et al, Am J Physiol Cell Physiol, 286(5): C987-C997 (2004). Like other cadherins, VE-cadherin mediates calcium-dependent, homophilic adhesion and functions as a plasma membrane attachment site for the cytoskeleton. However, VE-cadherin is integrated into signaling pathways and cellular systems uniquely important to the vascular endothelium. Recent advances in endothelial cell biology and physiology reveal properties of VE-cadherin that may be unique among members of the cadherin family of adhesion molecules. For these reasons, VE-cadherin represents a cadherin that is both prototypical of the cadherin family and yet unique in function and physiological relevance. Evidence is accumulating that the VE-cadherin-mediated cell-cell adhesion is controlled by a dynamic balance between phosphorylation and dephosphorylation of the junctional proteins including cadherins and catenins. Increased tyrosine phosphorylation of beta-catenin resulted in a dissociation of the catenin from cadherin and from the cytoskeleton, leading to a weak adherens junction (AJ). Similarly, tyrosine phosphorylation of VE-cadherin and beta-catenin occurred in loose AJ and was notably reduced in tightly confluent monolayers (Tinsley et al., J Biol Chem, 274, 24930-24934 (1999).

In addition the correct clustering of VE-cadherin monomers in adherens junctions is indispensable for a correct signalling activity of VE-cadherin, since cell bearing a chimeric mutant (IL2-VE) containing a full-length VE-cadherin cytoplasmic tail is unable to cause a correct signalling despite its ability to bind to beta-catenin and p120 (Lampugnani et al, Mol. Biol. of the Cell, 13, 1175-1189 (2002).

### Rho- and Rac-GTPases and Vascular Permeability

Rho GTPases are a family of small GTPases with profound actions on the actin cytoskeleton of cells. With respect to the functioning of the vascular system they are involved in the regulation of cell shape, cell contraction, cell motility and cell adhesion. The three most prominent family members of the Rho GTPases are RhoA, Rac and cdc42. Activation of RhoA induces the formation of f-actin stress fibres in the cell, while Rac and cdc42 affect the actin cytoskeleton by inducing membrane ruffles and microspikes, respectively (Hall, Science, 279:509-514.1998). While Rac and cdc42 can affect MLCK activity to a limited extent via activation of protein PAK (Goeckeler et al. J. Biol. Chem., 275, 24, 18366-18374 (2000), RhoA has a prominent stimulatory effect on actin-myosin interaction by its ability to stabilize the phosphorylated state of MLC (Katoh et al., Am. J. Physiol. Cell. Physiol. 280, C1669-C1679 (2001). This occurs by activation of Rho kinase that in its turn inhibits the phosphatase PP1M that hydrolyses phosphorylated MLC. In addition, Rho kinase inhibits the actin-severing action of cofilin and thus stabilizes f-actin fibres (Toshima et al., Mol. Biol. of the Cell. 12, 1131-1145 (2001). Furthermore, Rho kinase can also be involved in anchoring the actin cytoskeleton to proteins in the plasma membrane and thus may potentially act on the interaction between junctional proteins and the actin cytoskeleton (Fukata et al. Cell Biol 145:347-361 (1999).

Thrombin can activate RhoA via Gα12/13 and a so-called guanine nucleotide exchange factor (GEF) (Seasholtz et al; Mol: Pharmacol. 55, 949-956 (1999). The GEF exchanges RhoA-bound GDP for GTP, by which RhoA becomes active. By this activation RhoA is translocated to the membrane, where it binds by its lipophilic geranyl-geranyl-anchor.

RhoA can be activated by a number of vasoactive agents, including lysophosphatidic acid, thrombin and endothelin. The membrane bound RhoA is dissociated from the membrane by the action of a guanine dissociation inhibitor (GDI) or after the action of a GTPase-activating protein (GAP). The guanine dissociation inhibitors (GDIs) are regulatory proteins that bind to the carboxyl terminus of RhoA.

GDIs inhibit the activity of RhoA by retarding the dissociation of GDP and detaching active RhoA from the plasma membrane. Thrombin directly activates RhoA in human endothelial cells and induces translocation of RhoA to the plasma membrane. Under the same conditions the related GTPase Rac was not activated. Specific inhibition of RhoA by C3 transferase from Clostridium botulinum reduced the thrombin-induced increase in endothelial MLC phosphorylation and permeability, but did not affect the transient histamine-dependent increase in permeability (van Nieuw Amerongen et al. Circ Res. 1998; 83:1115-11231 (1998). The effect of RhoA appears to be mediated via Rho kinase, because the specific Rho kinase inhibitor Y27632 similarly reduced thrombin-induced endothelial permeability.

Rac1 and RhoA have antagonistic effects on endothelial barrier function. Acute hypoxia inhibits Rac1 and activates RhoA in normal adult pulmonary artery endothelial cells (PAECs), which leads to a breakdown of barrier function (Wojciak-Stothard and Ridley, Vascul Pharmacol., 39:187-99 (2002). PAECs from piglets with chronic hypoxia induced pulmonary hypertension have a stable abnormal phenotype with a sustained reduction in Rac1 and an increase in RhoA activity. These activities correlate with changes in the endothelial cytoskeleton, adherens junctions and permeability. Activation of Rac1 as well as inhibition of RhoA restored the abnormal phenotype and permeability to normal (Wojciak-Stothard et al., Am. J. Physiol, Lung Cell Mol. Physiol. 290, L1173-L1182 (2006).

It is therefore desirable to screen for substances that restore the physiologic balance of Rac1 and RhoA activity to a level that is observed in endothelial cells in normal and stable conditions. Preferably this effect is caused by a stabilization of the clustering of VE-cadherin in the adherens junction.

### Focal Adhesion Kinase and its Role in Endothelial Permeability

Focal adhesion kinase is composed of a central catalytic domain flanked by large N- and C-terminal domains. The N-terminal region contains the FERM homology that can bind integrins and growth factor receptors. The non-catalytic domain in the C-terminal, also referred to as FRNK(FAK-related non-kinase), carries the FATsequence which not only directs FAK to adhesion complexes for signalling, but also provides binding sites for other docking molecules to interact with the cytoplasmic To date, at least five tyrosine residues have been identified in FAK.

Phosphorylation of these tyrosine residues directly correlates with the kinase activity. This is supported by a reciprocal relationship between FAK activity and monolayer permeability. Several models have been proposed to explain the effect of focal adhesion formation on the barrier structure. The increased adhesion of endothelial cells to the extracellular matrix may help to stabilize monolayers against detachment due to the lateral contractile forces produced by inflammatory mediators. Thus, focal adhesion activation may occur in parallel with cell contraction to compensate for the diminished cell-cell binding during inflammatory stimulation. Another hypothesis proposes that FAK activation and focal adhesion reorganization actively contribute to the opening of endothelial cell-cell junctions by providing a mechanical basis for endothelial cells to contract or change shape. The last, but not the least, possibility is that the focal complex serves as a point of convergence for multiple scaffold proteins or signalling molecules to be integrated, which in turn affect the barrier function.

In addition to the well-characterized activation of MAPK, PI3K, and eNOS, potential signalling events downstream from FAK include the myosin light chain phosphorylation-triggered actin-myosin contraction and Rho-dependent stress fiber formation which are characteristic features of paracellular permeability (Wu, J Physiol 569, 359-366 (2005). It is therefore desirable to inhibit FAK phosphorylation in order to promote endothelial integrity.

### Inflammation and Endothelial Dysfunction: Pathological and Therapeutic Consequences

Endothelial dysfunction and leakiness of the endothelial barrier is an important component of a range of inflammatory diseases. The inflammatory response is characterized by an extravasation of blood constituents such as plasma proteins and of blood serum leading to severe interstitial tissue edema.

In addition, neutrophils, which are the primary agents of the inflammatory response, are able to emigrate from the blood stream into the underlying tissue. Together, these effects of endothelial layer leakiness cause substantial damage to healthy organs and tissues. They have been implicated in organ damage of a number of diseases including, but not limited to adult respiratory distress syndrome (ARDS), acute lung injury (ALI), glomerulonephritis, acute and chronic allograft rejection, inflammatory skin diseases, rheumatoid arthritis, asthma, atherosclerosis, systemic lupus erythematosus (SLE), connective tissue diseases, vasculitis, as well as ischemia-reperfusion injury in limb replantation, myocardial infarction, crush injury, shock, stroke and organ transplantation. It is also a prerequisite for new blood vessel formation by proliferation of endothelial and therefore can result in disease where such angiogenesis has been shown to play a pathogenetic role, including but not limited to wet age-related macula degeneration and cancer progression and metastasis.

### BRIEF DESCRIPTION OF THE INVENTION

The present invention is directed to methods for screening, identification, characterization and use of proteins, peptides, peptidomimetics, antibodies and small molecules that modulate interactions and signalling events mediated by agents that cause endothelial hyperpermeability. The agents identified by these screening methods exert their effect by binding to and modulating the conformation and/or phosphorylation status of vascular endothelial (VE)-cadherin expressed in the adherens junctions of endothelial cell layers. More specifically these agents promote endothelial integrity by stabilizing the clustering of VE-cadherin at intercellular junctions. Given the importance of disruption of the endothelial barrier function for a broad range of diseases, these agents have broad applicability as therapeutic and/or prophylactic medicinal products.

According to one aspect, the present invention provides a method of screening for proteins, peptides, peptidomimetics, antibodies or small organic molecules that increase the activity of Rac1 by virtue of their binding to the extracellular portion of this protein, the method comprising the steps of:
a. contacting a confluent layer of cultured endothelial cells with at least one of the test compounds
b. lysing the endothelial cells with a lysation buffer
c. measuring the amount of Rac1 activity with a specific assay.

In another embodiment, the present invention provides a method for screening of proteins, peptides, peptidomimetics, antibodies and small organic molecules that prevent the activation of RhoA and consequentially the change in the cytoskeletal structure of the endothelial cells, the method comprising the steps of:
a. contacting a confluent layer of cultured endothelial cells with thrombin in the presence of at least one of the test compounds
b. lysing the endothelial cells with a lysation buffer
c. measuring the RhoA activity with a specific assay.

In another embodiment, the present invention provides a method for screening of proteins, peptides, peptidomimetics, antibodies and small organic molecules that prevent the phosphorylation of focal adhesion kinase, the method comprising the steps of:
a. contacting a confluent layer of cultured endothelial cells with thrombin in the presence of at least one of the test compounds
b. lysing the endothelial cells with a lysation buffer
c. measuring the phosphorylation of focal adhesion kinase with a specific assay.

In another embodiment, the present invention provides a method for screening of proteins, peptides, peptidomimetics, antibodies and small organic molecules that prevent vascular leak in a warm-blooded animal undergoing systemic inflammatory response, the method comprising the following steps:
a. Initiation of a systemic inflammatory response by applying an appropriate dose of bacterial lipopolysaccharide (LPS)
b. Exposing the animal to at least one of the test compounds
c. injecting the animal with an appropriate amount of fluorescence labelled micro-beads of appropriate size
d. sacrificing the animal after an appropriate time period
e. excising and homogenizing an organ or tissue of the animal
f. measuring the amount of fluorescence in the homogenate.

### BEST METHOD OF CARRYING OUT THE INVENTION

Preferentially, these assays are performed in the sequence described above, which constitutes a screening tree to selectively identify compounds with the specific physiological activities claimed by the current invention.

The useful methods of analysing the activation status of Rac1 and RhoA are based on the principle of the so-called pull down assay. In this format, the GTP-bound active state of the respective protein in a cell lysate is bound to an immobilized binding partner and detected with a monoclonal antibody (MAb) specifically directed against the protein in question. The amount of the GTP-bound active state can subsequently be quantified through suitable detection methods, including but not limited to Western blotting or luminescence detection.

For measuring the activation of Rac1 in human umbilical vein endothelial cells (HUVECs), the cells are incubated with the test compound under suitable conditions for various periods of time up to 30 min and lysed afterwards. The lysate is added to a suitably immobilized p21-binding domain (PBD) of p21-activated protein kinase (PAK) and the amount of activated Rac1 is quantified with a Rac1-specific MAb.

For measuring the inhibition of thrombin induced activation of RhoA in HUVECs, the cells are incubated under suitable conditions with a suitable amount of thrombin with and without the test compound for various periods of time up to 10 min and lysed afterwards. The lysate is added to a suitably immobilized rhotekin-binding domain (RBD) and the signal measured with an appropriate detection method.

For measuring the inhibition of thrombin meditated FAK phosphorylation in HUVECs, the cells are incubated under suitable conditions with a suitable amount of thrombin with and without the test compound for various periods of time up to 60 min and lysed afterwards lysates were subjected to SDS-PAGE and western blot analysis with site-specific antibodies directed against FAK phosphorylated tyrosine residues.

For measuring the inhibition of LPS-induced vascular leak in rodents, the animals receive injections of amounts of gram-negative lipopolysaccharide (LPS) suitable to achieve a systemic inflammatory response. After various periods of time between 0 and 4 hours, the test substance is injected intravenously, followed by the injection of a suitable amount of fluorescent microbeads. Subsequently the animals are sacrificed, and organs (lung, kidney, spleen, heart, brain) are excised and cut into thin slices suitable for microscopic analysis and fixated with paraformaldehyde. The numbers of extravasated microspheres are counted using a fluorescent microscope.

Alternatively, the organ are homogenized and the amount of micro beads trapped in the tissues are measured using a suitable fluorescence detection device.

The compounds identified with the screening methods according to the present invention are useful for development of drugs for the prevention and/or treatment of diseases which are caused by an inflammatory reaction and/or endothelial disruption and vascular leak. Therefore, according to another embodiment of the current invention, the compounds of the present invention are administered for treatment and/or prevention of, but not restricted to, septic shock, wound associated sepsis, post-ischemic reperfusion injury, such as after myocardial infarction/reperfusion or organ transplantation), frost-bite injury or shock, acute inflammation mediated lung injury, such as respiratory distress syndrome, acute pancreatitis, liver cirrhosis, uveitis, asthma, traumatic brain injury, nephritis, atopic dermatitis, psoriasis, inflammatory bowel disease, macula degeneration of the eye, diabetic retinopathy, neovascular glaucoma, retinal vein occlusion and tumour progression. In order to achieve their therapeutic effects in these diseases, the compounds of the present invention may be given orally or parenterally and maybe be formulated into suitable pharmaceutical formulation with pharmaceutically acceptable excipients or carriers.

The present invention therefore also relates to a pharmaceutical composition containing an active ingredient identified by the method of screening according to the present invention and further comprising pharmaceutically acceptable excipients or carriers.

Pharmaceutically acceptable excipients are those which are approved by a regulatory agency of the Federal or State governments or listed in the U.S. Pharmacopeia or any other generally recognized pharmacopeia for use in animals, and more particularly in humans. Such pharmaceutical carriers can be sterile liquids, such as water and oils, including those of animal, vegetable and synthetic origin, e.g. peanut oil, soybean oil, mineral oil and the like. Aqueous carriers nay contain for instance also contain dextrose or glycerol.

Suitable excipients may include, but are not restricted to, starch, glucose, lactose, sucrose, gelatin, silica gel, sodium stearate, glycerol monostearate, talc, sodium chloride, glycerol, propylene glycol, ethanol and the like. The composition may also include wetting and/or emulsifying agents, or pH buffering agents.

The compositions can take the form of solutions, suspensions, emulsions, tablets, capsules, powders, or slow release formulations. Such compositions will contain a therapeutically effective amount of the compound together with a suitable amount of carrier so as to provide a form for proper administration to the subject to be treated and suitable for the form of treatment.

### BRIEF DESCRIPTION OF THE FIGURES

FIG. 1 Compounds identified through the claimed method of screening

FIG. 2 Activation of Rac1 with compound 1A. This gel is the result of a pull-down assay as described in Example 1. Lane 1: medium control, lane 2: HUVEC activation with thrombin for 1 min, lane 3: treatment with compound 1A alone for 1 min; lane 4: HUVEC with thrombin and compound 1A for 1 min, lane 5: thrombin for 5 min; lane 6: compound 1A for 5 min, lane 7: thrombin and compound 1A for 5 min. Beta-actin was used to control for total protein content.

FIG. 3 Inhibition of thrombin induced activation of RhoA by compound 1A. This gel is the result of a pull-down assay as described in Example 1. Lane 1: medium control, lane 2: HUVEC activation with thrombin for 1 min, lane 3: treatment with compound 1A alone for 1 min; lane 4: HUVEC with thrombin and compound 1A for 1 min, lane 5: thrombin for 5 min; lane 6: compound 1A for 5 min, lane 7: thrombin and compound 1A for 5 min. Beta-actin was used to control for total protein content.

FIG. 4 Activation of Rac1 with compound 1B. This gel is the result of a pull-down assay as described in Example 1. Lane 1: medium control, lane 2: HUVEC activation with thrombin for 1 min, lane 3: treatment with compound 1B alone for 1 min; lane 4: HUVEC with thrombin and compound 1B for 1 min, lane 5: thrombin for 5 min; lane 6: compound 1B for 5 min, lane 7: thrombin and compound 1B for 5 min.

FIG. 5 Inhibition of thrombin induced activation of RhoA by compound 1B at time points 1, 5 and 10 min after stimulation with thrombin.

FIG. 6 Quantification and time dependency of activation of Rac1 by compound 1B

FIG. 7 Quantification and time dependence of inhibition of thrombin induced RhoA activation by compound 1B

FIG. 8 Inhibition of thrombin induced phosphorylation of FAK by compound 1A. This graphs shows the time-course of inhibition of phosphorylation of FAK induced by thrombin.

FIG. 9 Inhibition of LPS induced vascular leak by compound 1A. These are fluorescent images of lung slices from rats, in which vascular leak was induced by LPS treatment. Slice a) is from a control animal, slice b) is from an animal treated with compound 1A

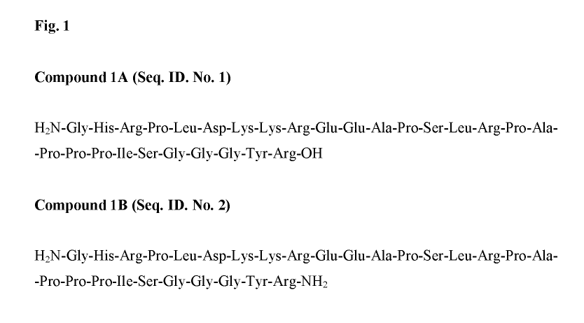

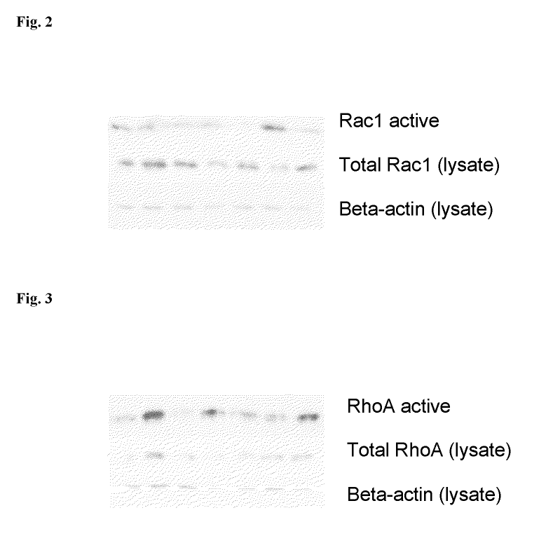

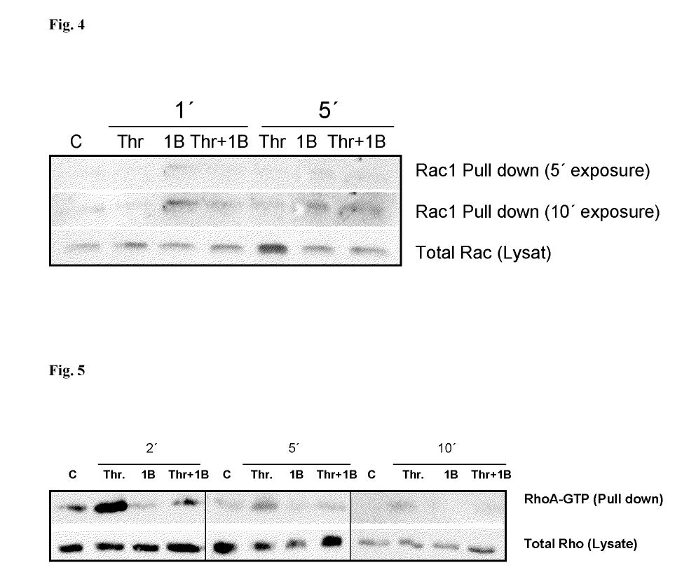

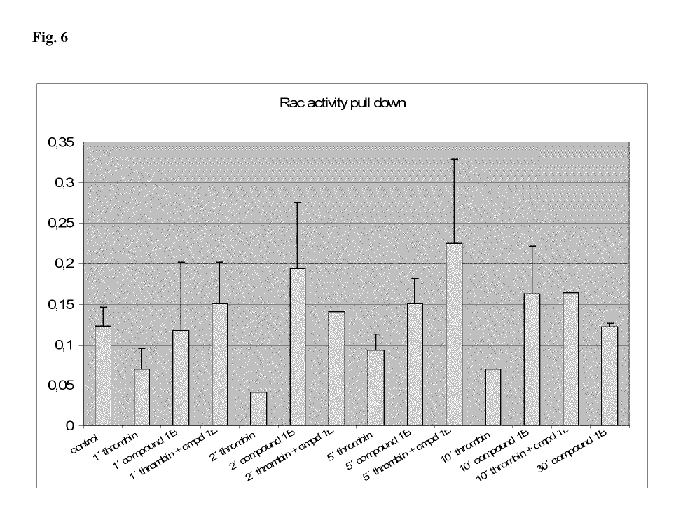

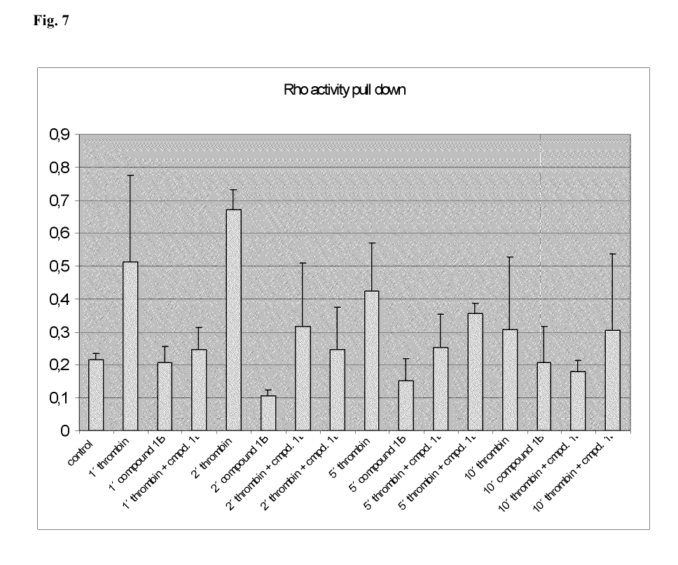

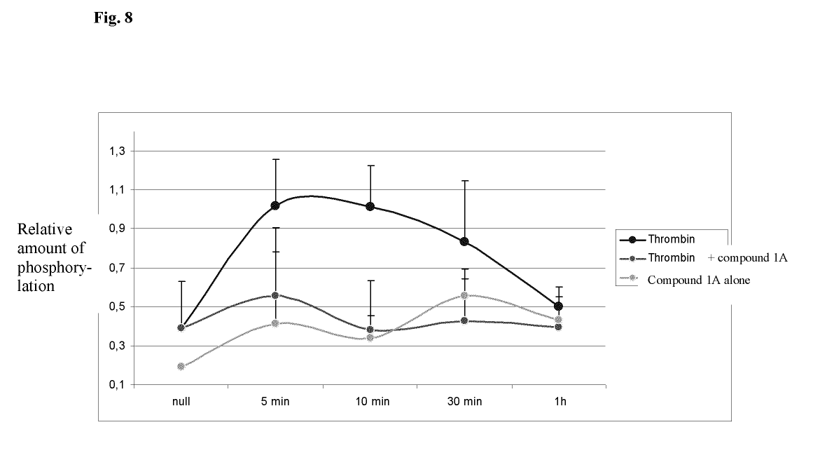

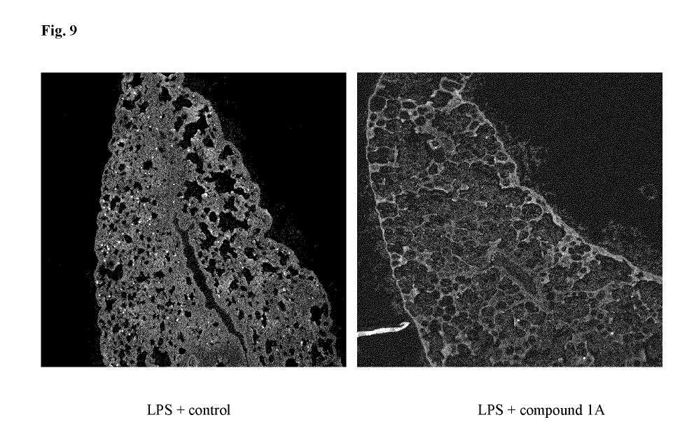

### EXAMPLE 1

### A Compound Screening Method for Identification of Substances Activating Rac1 Through Stabilization of VE-Cadherin Junctions

HUVECs are grown to confluence under standard conditions. Before induction of Rac1 activity HUVECs were starved for 4 h by using IMDM (Gibco) without growth factor and serum supplements. Rac1 activity is induced by adding 50 μg/ml of test compound into starvation medium for 1, 5 and 10 min. Active Rac1 was isolated using Rac1/Cdc42 Assay Reagent from Upstate according to manufactures instructions. Isolates were separated on a 15% polyacrylamid gel and blotted on Nitrocellulose-Membranes (Bio-Rad). Rac1 was detected by using Anti-Rac1 clone23A8, anti-mouse from Upstate (1:250).

Relative values compared to unstimulated control

| | |
|---|---|
| Control peptide 1 min | 1 |
| Control peptide 5 min | 1 |
| Control peptide 10 min | 1 |
| Compound 1B, 1 min | 2 +/− 0.2* |
| Compound 1B, 5 min | 2 +/− 0.1* |
| Compound 1B, 10 min | 1 +/− 0.1 |
| thrombin 1 min | 0.5 +/− 0.2* |
| thrombin 5 min | 0.5 +/− 0.2* |
| thrombin 10 min | 1 +/− 0.1 |
| thrombin + compound 1B, 1 min | 1 +/− 0.2# |
| thrombin + compound 1B, 5 min | 1 +/− 0.1# |
| thrombin + compound 1B, 10 min | 1 +/− 0.1 |

*denotes p < 0.05 compared to control
#denotes p < 0.05 between thrombin and thrombin + compound 1B

### EXAMPLE 2

### A Compound Screening Method for Identification of Substances Inhibiting Thrombin Induced RhoA Activation

HUVEC are grown to confluence under standard conditions. Before induction of Rho activity HUVEC were starved for 4 h by using IMDM (Gibco) without growth factor and serum supplements. After the starvation period 5 U/ml Thrombin (Calbiochem) or 5 U thrombin plus 50 μg/ml of test compound are added to the starvation medium for 1, 5 and 10 min. Active RhoA was isolated using Rho Assay Reagent from Upstate according to manufactures instructions. Isolates were separated on a 15% polyacrylamid gel and blotted on Nitrocellulose-Membrane (Bio-Rad). RhoA was detected by using Anti-Rho (-A, -B, -C), clone55 from Upstate (1:500).

Relative values compared to unstimulated control

| | |
|---|---|
| Control peptide 1 min | 1 |
| Control peptide 5 min | 1 |
| Control peptide 10 min | 1 |
| Compound1B, 1 min | 1 +/− 0.2 |
| Compound 1B 5 min | 1 +/− 0.1 |
| Compound 1B 10 min | 1 +/− 0.1 |
| thrombin 1 min | 2.5 +/− 0.2* |
| thrombin 5 min | 2.5 +/− 0.2* |
| thrombin 10 min | 1 +/− 0.2 |
| thrombin + compound 1B 1 min | 1 +/− 0.3# |
| thrombin + compound 1B 5 min | 1 +/− 0.1# |
| thrombin + compound 1B 10 min | 1 +/− 0.1 |

*denotes p < 0.05 compared to control
#denotes p < 0.05 between thrombin and thrombin + compound 1B

### EXAMPLE 3

### A Compound Screening Method for Identification of Substances Inhibiting the Thrombin Induced FAK Phosphorylation at Autophosphorylation Site Tyr397

### Immunoprecipitation:

HUVEC were incubated with FX06 (50 μg/ml), Thrombin (1 U/ml, Sigma Aldrich) and Thrombin/test compound for indicated time points. After washing with ice cold PBS (GIBCO), cells were scrapped in Tris-lysis buffer (plus 1% Triton X (Bio-Rad), NP40 (Sigma Aldrich) and proteinase and phosphatase inhibitory cocktails (Sigma Aldrich)) from culture flasks and lysed for 20 min on ice. Lysates were heavily vortexed every 5 min. After lysis lysates were centrifuged (15.000 rpm/10 min/4° C.) and supernatants were added to 50 μl sepahrose beads (Sigma Aldrich) preincubated with 1 μg total FAK antibody (BD Transduction Laboratories). Beads were agitated on the wheel for 2 h at 4° C., followed by 3 times washing with ice cold PBS, the addition of 2× sample buffer and incubation at 95° C. for 5 min. The sample buffer was then removed from the beads and applied to western blotting.

### Western Blot:

10% polyacrylamide gels were run for separating precipitated proteins. Gels were blotted onto PVDF (Bio-Rad) membranes using the hoefer semi dry blotting system. Membranes were then washed with TBS/0.5% TWEEN (TBST), blocked with 1% BSA/TBST for 1 h at RT and then incubated with the p397 FAK antibody (0.2 μg/ml; BD Transduction Laboratories) in 1% BSA/TBST over night at 4° C. For detection, a HRP-labeled goat anti-mouse Ab (1:25 000; Bio-Rad) in TBST was used and bound Abs were visualized by chemiluminescence (ECL-system, Amersham Corp., Arlington Heights, Ill.) and recorded on film.

Relative values compared to unstimulated control

| | |
|---|---|
| Control peptide 1 min | 1 |
| Control peptide 5 min | 1 |
| Control peptide 10 min | 1 |
| Compound1A, 1 min | 5.5 +/− 0.2* |
| Compound 1A, 5 min | 2 +/− 0.1* |
| Compound 1A, 10 min | 1 +/− 0.1 |
| thrombin 1 min | 4.5 +/− 0.2* |
| thrombin 5 min | 4.5 +/− 0.2* |
| thrombin 10 min | 3.8 +/− 0.1* |
| thrombin + compound 1A, 1 min | 3 +/− 0.5* |
| thrombin + compound 1A, 5 min | 2 +/− 0.1*# |
| thrombin + compound 1A, 10 min | 1.3 +/− 0.1*# |

*denotes p < 0.05 compared to control
#denotes p < 0.05 between thrombin and thrombin + FX06

### EXAMPLE 4

### A Compound Screening Method for Identification of Substances Inhibiting the LPS Induced Vascular Leak in Rodents

Male Him OFA/SPF rats (Institute for Biomedical Research, Medical School Vienna) with a body weight of 260-320 g are housed at the Institute for Biomedical Research, Medical School Vienna. All experiments were approved by Amt der Wiener Landesregierung, MA58. Rats are anaesthetised with 100 mg/kg sodium thiopentone (Sandoz). The trachea is cannulated to facilitate respiration. The right jugular vein is cannulated for the administration of drugs. To measure the Mean Arterial Blood Pressure (MAP) a catheter is placed into the right carotid artery. After surgery the animals are randomized in treatment groups. All rats receive a fluid replacement (600 μl 0.9% saline as an i.v. infusion) and are allowed to stabilize for 15 min. Body temperature is controlled with a homeothermic blanket throughout the whole experiment. After the stabilisation period, the endotoxic shock is induced by a bolus injection of 12 mg/kg LPS (E. coli serotype 0.127:B8; Sigma). 60 min after LPS administration the animals receive a bolus injections of 3 mg/kg of test compound or saline. 5 h 50 min after the LPS administration the rats receive an bolus injection of fluorescent microspheres; 125×10⁶ beads/kg body weight (Fluo Spheres Polystyrene Microspheres; 1 μm yellow-green fluorescent (505/515) Invitrogen Molecular Probes)

6 h after LPS administration the animals are sacrificed and the lungs are removed to assess vascular leakage. Vascular leakage of the lung is assessed by measurement of the fluorescence per g of tissue. For these purpose the lung tissue was digested with ethanolic KOH and the fluorescent microspheres are recovered by sedimentation as recommended by the “Manual for using Fluorescent Microspheres to measure organ perfusion” Fluorescent Microsphere Resource Center; University of Washington. Fluorescence is measured using a Spectra Max Gemini S Fluorometer

Relative fluorescence within lungs of LPS-treated animals

| | |
|---|---|
| sham | 656 +/− 210 |
| LPS | 3454 +/− 790* |
| LPS + compound 1A | 2275 +/− 795*# |

*denotes p < 0.05 compared to control
#denotes p < 0.05 between thrombin and thrombin + compound1A

1. A method of screening for compounds out of the group consisting of proteins, peptides, peptidomimetics, antibodies and small organic molecules that increase the activity of the protein Rac1 by virtue of their binding to the extracellular portion of this protein, the method comprising the steps of:
a. contacting a confluent layer of cultured endothelial cells with at least one of test compounds,
b. lysing the endothelial cells with a lysation buffer, and
c. measuring the amount of Rac1 activity.

2. A method for screening for compounds out of the group consisting of proteins, peptides, peptidomimetics, antibodies and small organic molecules that prevent the activation of RhoA and consequentially the change in the cytoskeletal structure of endothelial cells, the method comprising the steps of:
a. contacting a confluent layer of cultured endothelial cells with thrombin in the presence of at least one of test compounds,
b. lysing the endothelial cells with a lysation buffer, and
c. measuring the RhoA activity.

3. A method for screening for compounds out of the groups consisting of proteins, peptides, peptidomimetics, antibodies and small organic molecules that prevent the phosphorylation of focal adhesion kinase, the method comprising the steps of:
a. contacting a confluent layer of cultured endothelial cells with thrombin in the presence of at least one of the test compounds,
b. lysing the endothelial cells with a lysation buffer, and
c. measuring the phosphorylation of focal adhesion kinase.

4. A method for screening for compounds out of the group consisting of proteins, peptides, peptidomimetics, antibodies and small organic molecules that prevent vascular leak in a warm-blooded animal undergoing systemic inflammatory response, the method comprising the following steps:
a. initiation of a systemic inflammatory response by applying an appropriate dose of bacterial lipopolysaccharide (LPS),
b. exposing the animal to at least one of the test compounds,
c. injecting the animal with an appropriate amount of fluorescence labelled micro-beads of appropriate size,
d. sacrificing the animal after an appropriate time period,
e. excising and homogenizing an organ or tissue of the animal, and
f. measuring the amount of fluorescence in the homogenate.

5. A method for screening for compounds out of the group consisting of proteins, peptides, peptidomimetics, antibodies and small organic molecules, characterized in that it comprises the method according to claim 1.

6. A method for screening for compounds out of the group consisting of proteins, peptides, peptidomimetics, antibodies and small organic molecules, characterized in that it comprises the method according to claim 2.

7. The method for screening according to claim 5, wherein the following method steps are carried out subsequently;
in a method for screening for compounds out of the group consisting of proteins, peptides, peptidomimetics, antibodies and small organic molecules that prevent the activation of RhoA and consequentially the change in the cytoskeletal structure of endothelial cells:
a. contacting a confluent layer of cultured endothelial cells with thrombin in the presence of at least one of test compounds
b. lysing the endothelial cells with a lysation buffer, and
c. measuring the RhoA activity.

8. Compounds out of the group consisting of proteins, peptides, peptidomimetics, antibodies and small organic molecules identified with the screening method according to claim 1 for development of drugs for the prevention and/or treatment of diseases which are caused by an inflammatory reaction and/or endothelial disruption and vascular leak.

9. Pharmaceutical preparation containing compounds according to claim 8.

10. Pharmaceutical preparation according to claim 9 for treatment and/or prevention of septic shock, wound associated sepsis, post-ischemic reperfusion injury, such as after myocardial infarction/reperfusion or organ transplantation), frost-bite injury or shock, acute inflammation mediated lung injury, such as respiratory distress syndrome, acute pancreatitis, liver cirrhosis, uveitis, asthma, traumatic brain injury, nephritis, atopic dermatitis, psoriasis, inflammatory bowel disease, macula degeneration of the eye, diabetic retinopathy, neovascular glaucoma, retinal vein occlusion and tumour progression.

11. A method for screening for compounds out of the group consisting of proteins, peptides, peptidomimetics, antibodies and small organic molecules, characterized in that it comprises the method according to claim 3.

12. A method for screening for compounds out of the group consisting of proteins, peptides, peptidomimetics, antibodies and small organic molecules, characterized in that it comprises the method according to claim 4.

13. The method for screening according to claim 5, wherein the following method steps are carried out subsequently:
in a method for screening for compounds out of the groups consisting of proteins, peptides, peptidomimetics, antibodies and small organic molecules that prevent the phosphorylation of focal adhesion kinase,
a. contacting a confluent layer of cultured endothelial cells with thrombin in the presence of at least one of the test compounds,
b. lysing the endothelial cells with a lysation buffer, and
c. measuring the phosphorylation of focal adhesion kinase.

14. The method for screening according to claim 5, wherein the following method steps are carried out subsequently:
In a method for screening for compounds out of the group consisting of proteins, peptides, peptidomimetics, antibodies and small organic molecules that prevent vascular leak in a warm-blooded animal undergoing systemic inflammatory response,
a. initiation of a systemic inflammatory response by applying an appropriate dose of bacterial lipopolysaccharide (LPS),
b. exposing the animal to at least one of the test compounds,
c. injecting the animal with an appropriate amount of fluorescence labelled micro-beads of appropriate size,
g. sacrificing the animal after an appropriate time period,
h. excising and homogenizing an organ or tissue of the animal, and measuring the amount of fluorescence in the homogenate.

15. Compounds out of the group consisting of proteins, peptides, peptidomimetics, antibodies and small organic molecules identified with the screening method according to claim 2 for development of drugs for the prevention and/or treatment of diseases which are caused by an inflammatory reaction and/or endothelial disruption and vascular leak.

16. Compounds out of the group consisting of proteins, peptides, peptidomimetics, antibodies and small organic molecules identified with the screening method according to claim 3 for development of drugs for the prevention and/or treatment of diseases which are caused by an inflammatory reaction and/or endothelial disruption and vascular leak.

17. Compounds out of the group consisting of proteins, peptides, peptidomimetics, antibodies and small organic molecules identified with the screening method according to claim 4 for development of drugs for the prevention and/or treatment of diseases which are caused by an inflammatory reaction and/or endothelial disruption and vascular leak.
